(19) United States
(12) Patent Application Publication
(10) Pub. No.: US 2005/0074784 A1
(43) Pub. Date: Apr. 7, 2005

(54) Integrated biochip with continuous sampling and processing (CSP) system

(76) Inventor: Tuan Vo-Dinh, Knoxville, TN (US)

Correspondence Address: Akerman Senterfitt;4th Floor, 222 Lakeview Avenue, P.O. Box 3188, West Palm Beach, FL, 33402-3188, US

(21) Appl. No.: 10/680,046

(22) Filed: Oct. 7, 2003

(51) Int. Cl. C12Q 1/68; G01N 33/53; C12M 1/34

(52) U.S. Cl. 435/6; 435/7.1; 435/287.2

(57) ABSTRACT

An integrated circuit based analyte detection system includes a plurality of bioprobe microarrays, each of the microarrays having a plurality of probe elements for combining with at least one target molecule. The probe elements generate an identifiable signal when combined with target molecules responsive to incident electromagnetic radiation. Structure is provided for translating the microarrays, allowing microarrays used by the system to be replaced by other microarrays. An integrated circuit microchip includes a plurality of detection channels to which the probe elements are brought into optical alignment.

### STATEMENT REGARDING FEDERALLY SPONSORED RESEARCH OR DEVELOPMENT

The United States Government has rights in this invention pursuant to Contract No. DE-AC05-00OR22725 between the United States Department of Energy and UT-Battelle, LLC.

### CROSS-REFERENCE TO RELATED APPLICATIONS

Not applicable.

### FIELD OF THE INVENTION

The invention relates to systems and methods for chemical and biological agent identification, particularly chip-based sensors which provide continuous sampling and processing.

### BACKGROUND OF THE INVENTION

There is a strong interest in the development of improved biosensors for environmental and biomedical diagnostics. Detection technology is needed for the rapid and continuous identification and quantification of the entire suite of chemical/biological (CB) agents as well as the detection of precursors, degradation products and solvents associated with their manufacture and distribution. For example, counter-terrorism could be aided by a system capable of detecting of a wide variety of known CB agents on a real-time or near real-time basis.

### SUMMARY OF INVENTION

An integrated circuit-based detection system includes a plurality of probe microarrays. Each microarray has a plurality of probes for combining with at least one target molecule. Responsive to incident electromagnetic radiation the probes generate an identifiable signal when combined with the target molecule. Structure for translating the plurality of microarrays is provided. Translation of the microarrays permits a replenishable supply of probes to be provided, such as after a predetermined amount of time.

The ability to provide a replenishable supply of probes permits continuous sampling and processing, other than the brief periods of time during microarray translation required to replace one microarray with another microarray. An integrated circuit microchip including a plurality of detection channels to which the probe elements are brought into optical alignment provides sensing for the presence of the target molecule(s) based on the presence of, or absence of, the generated signal.

The microarrays can include at least one protein probe and at least one nucleic acid probe. In another embodiment, the microarrays can comprise at least two probe types selected from DNA, RNA, antibodies, proteins, enzymes, cells or cell components, and biomimetics. The biomimetics can be molecular imprint antibodies, DNA-based aptamers, PNA, cyclodextrins or dendrimers.

The system can include an air sampler for collecting airborne samples. In addition, the system can include a sample concentrator, such as a flow injection analysis system. The flow injection analysis system can comprise a plurality of microparticles coated with bioreceptors, the coated microparticles being mixed with the sample at the sample concentrator. In this embodiment, the sample concentrator preferably includes a size exclusion device for eliminating substances not trapped onto the coated microparticles. In an alternate embodiment, a continuous tape having a plurality of microarrays can provide sample collection and processing.

The system can include a biofluidics system having a plurality of microfluidic channels. The biofluidics system directs sample containing fluids through the microfluidic channels to the microarrays.

The plurality of microarrays can be provided on a translatable tape. In this embodiment, the system preferably includes a structure for translating the tape. In another embodiment, the microarrays are provided on a rotatable disk.

The integrated circuit microchip can provide a separate detector channels for each of the receptor probes on the microarrays. The detectors for the detector channels can be photodiodes or phototransistors, or other photodetectors.

The system preferably includes a target amplification system. The target amplification system can be PCR, SDA, ELISA or immuno-PCR. For DNA containing samples, the system preferably includes a lysis system.

The system can include an audio or visual display to indicate the presence of the target molecule. The system can also include structure for attaching the system to an individual. This embodiment permits realization of, for example, a real time or near-real time continuous and automated personal environmental monitoring system.

A method of detecting target analytes includes the steps of providing a plurality of probe microarrays, each of the microarrays having a plurality of probe elements for combining with at least one target molecule. The probe elements generating an identifiable signal when combined with the target molecule in response to incident electromagnetic radiation. A first of a plurality of microarrays are exposed to a sample suspected of containing the target and then irradiated with electromagnetic radiation. Based on the presence or absence of the identifiable signal, it determined whether the target is present. The first microarray is automatically replaced with an other of the plurality of microarrays, and the exposing step, irradiating and determining step are repeated with another of the microarrays.

### BRIEF DESCRIPTION OF THE DRAWINGS

A fuller understanding of the present invention and the features and benefits thereof will be accomplished upon review of the following detailed description together with the accompanying drawings, in which:

FIG. 1 illustrates a block diagram of an integrated biochip with a continuous sampling and processing (CSP) which includes a translatable tape for providing a continuous supply of receptor probes, according to an embodiment of the invention.

FIG. 2A illustrates a biochip system including a translatable tape which provides a replenishable supply of probes, according to an embodiment of the invention.

FIG. 2B illustrates a biochip system including a translatable tape which provides a replenishable supply of probes and includes a dichroic filter, according to another embodiment of the invention.

FIG. 3A illustrates a biochip system which includes a rotating disk for providing a replenishable supply of probes, according to another embodiment of the invention.

FIG. 3B illustrates a biochip system which includes a rotating disk for providing a replenishable supply of probes and includes a dichroic filter, according to another embodiment of the invention.

FIG. 4 illustrates a block diagram of an integrated biochip based system which includes a flow injection analysis system (FIA), according to an embodiment of the invention.

FIG. 5 illustrates steps in utilizing an exemplary CSP biochip which includes a flow injection analysis (FIA) system, according to another embodiment of the invention.

FIG. 6 illustrates a block diagram of an integrated CSP biochip including a replenishable first tape for sample collection and processing and a second tape for providing a replenishable supply of probes for analyte detection, according to an embodiment of the invention.

FIG. 7 illustrates an integrated CSP biochip including a multiplex tape for providing a replenishable supply of a variety of probes, according to an embodiment of the invention.

FIG. 8 illustrates and an integrated CSP biochip including a multiplex tape and multiplex PCR microchamber amplification system, according to an embodiment of the invention.

FIGS. 9A and B illustrate bioreceptor coated microstructures, while FIG. 9C-G illustrate various tape microstructures, according to another embodiment of the invention.

FIG. 10 shows a schematic diagram of a personal integrated CSP biochip system, according to another embodiment of the invention.

### DETAILED DESCRIPTION OF THE PREFERRED EMBODIMENTS

The system includes a fully integrated continuous sampling and processing (CSP) biochip-based system which can be used to simultaneously, continuously and automatically identify, and optionally quantify the concentration of, a diverse array of chemical and biological (CB) agents. As used herein, the phrase “continuous sampling” refers a system or method which can provide a replenishable supply of replacement receptor probes. For example, a replenished microarray of receptor probes may be provided to replace a given probe microarray following passage of a predetermined period of time of operation or following an indication that the receptor sites currently in service are occupied to a predetermined extent.

In one embodiment, microarrays can simultaneously provide different bioreceptor types, such as two or more of antibodies, DNA, enzyme and cell-based probes. PNA (polypeptides nucleic acid) may be used instead of, or in addition to, DNA probes. Thus, the invention can be used to simultaneously detect a plurality of diverse chemical and biological target analytes, such as, but not limited to chemical toxins, nucleic acids, proteins and pathogens, using a single device.

The inventor has disclosed advanced biochips in U.S. Pat. No. 6,197,503 (2001) to Vo-Dinh, et al, entitled “Integrated Circuit Biochip Microsystem Containing Lens,” and U.S. Pat. No. 6,448,064 to Vo-Dinh, et al. (2002) entitled “Integrated Circuit Biochip Microsystem”. These patents disclose chip-based biosensor systems including bioreceptor probes and CMOS based biochip sensing and processing circuitry, and are both hereby incorporated by reference into this application in their entirety.

The inventor has also disclosed a chip-based biochip device including a diverse variety of bioreceptor probe types (e.g. DNA, antibody and protein) on a single sampling platform (U.S. patent application Ser. No. 09/890,047) entitled “Multifunctional and Multispectral Biosensor Devices and Methods of Use”. The disclosed system permits simultaneous detection of a diverse group of target molecules. U.S. patent application Ser. No. 09/890,047 is hereby incorporated by reference into this application in its entirety.

Biosensors combine two important concepts that integrate “biological recognition” and “sensing”. The basic principle of a biosensor is to detect this molecular recognition and to transform it into another type of signal using a transducer. The selected transducer may produce either an optical signal (e.g. optical biosensors) or an electrochemical signal (e.g. electrochemical biosensors).

Construction of a biosensor generally involves the integration of several basic elements of very different natures. The basic steps include selection or development of the bioreceptor(s), selection of the excitation source, selection or development of the transducer, and integration of the excitation source-bioreceptor-transducer system. The role of the bioreceptor is to identify the chemical or biological target compounds via molecular recognition.

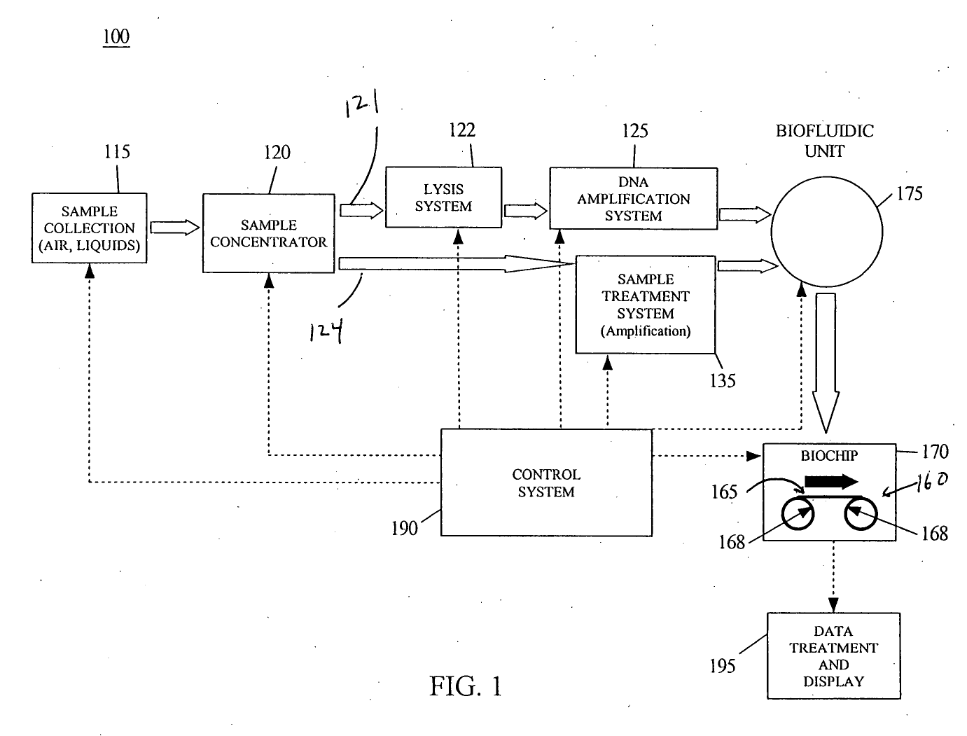

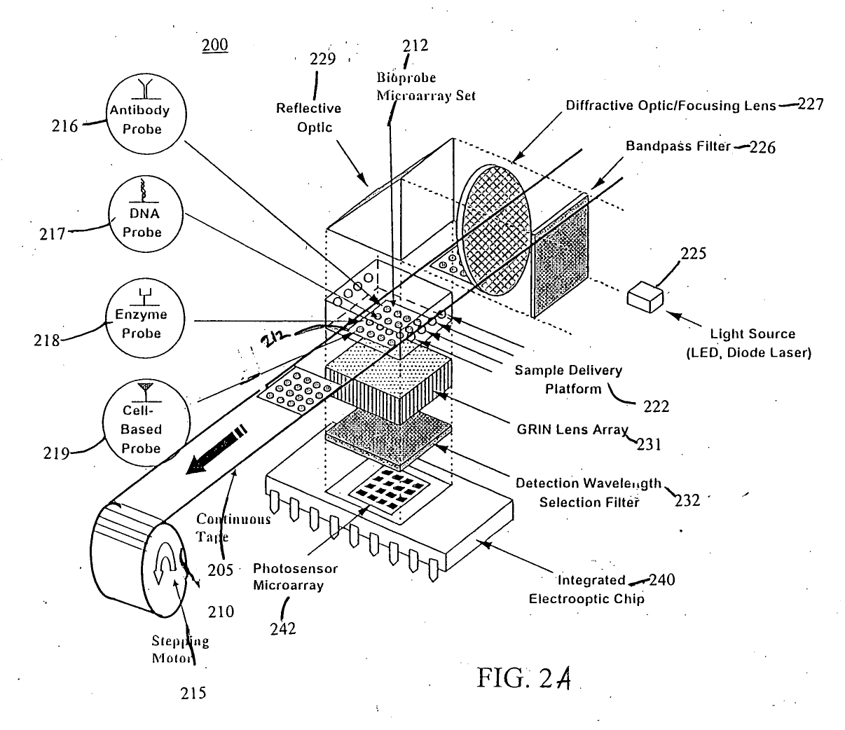

FIG. 1 shows one embodiment of a continuous sampling and process (CSP) biochip system 100 which comprises a continuous tape system 160, according to an embodiment of the invention. The continuous tape system 160 includes translatable tape 165 for providing a continuous supply of receptor probes and cassettes 168 for translating the tape. System 100 also includes a sample concentrator 120 and amplification system 125 and 135 for enhancing sensitivity of system 100 to permit detection and identification of target analytes at levels significantly lower than otherwise possible using earlier systems.

System 100 includes a sample collection device 115. Sample collection device 115 can comprise a flow injection system which is preferably compatible with microparticle-based substrates. An air sampler (Biocapture BT-550; Mesosystems Technology, Inc., Albuquerque, N.Mex.) can be used to collect samples from the air. The air sampler concentrates air particulates from the surrounding environment into a solvent solution of about 1-5 ml. If the sample to be monitored is a liquid sample, such as from a pharmaceutical process or an environmental waste stream, a portion of the liquid sample can be used directly thus avoiding the need for an air sampler.

The solution produced or collected sample collection device 115 can be further concentrated by sample concentrator 120 which can be based on several methods. One method comprises heating the sample to evaporate the solvent. Another method involves use of substrates coated with bioreceptors, such as antibodies or DNA, targeted to the species of interest. Since the multi-functional biochip can detect both DNA as well as proteins, samples can be simultaneously concentrated in both channels.

As shown in FIG. 1, the output of sample concentrator 120 is divided into two solution portions. A first portion 121 is sent to the DNA channel which comprises lysis system 122 followed by DNA amplification system 125, while a second portion 124 is sent to the non-DNA channel which comprises sample treatment system 135. The arrangement shown in FIG. 1 permits detection based on species of interest as well the DNA of those species.

The lysis system 122 of the DNA channel lyses sample solution components which may include bioagents, such as entire organisms, cells, and spores. Lysis system 122 can use heat, chemical, acoustic (ultrasound) or mechanical means to lyse the cells and release the DNA. In one embodiment, a bead beater device (e.g. VWR Company), which comprises a plurality of rapidly moving mechanical fingers move microbeads to break the spores and lyse the cells. The cellular DNA can then be amplified using polymer chain reaction (PCR) or other amplification techniques (e.g., strand displacement amplification (SDA) developed by BD Sciences). A commercial PCR device (Perkin Elmer PCR device) or a laboratory made PCR device can be used. A laboratory made PCR device can comprise thermoelectric blocs or Peltier chips (Advanced Thermoelectrics) for thermal cycling. With SDA, no thermal cycling is required and heater block or heating strips (Watlow, Inc.) can be used to maintain a constant temperature.

On the non-DNA channel(s), such as an antibody-based and/or protein-based detection channels, the sample is not lysed. Rather, the sample portion 124 is sent directly to a sample treatment 135, where amplification techniques such as ELISA can be used to enhance the concentration species of interest. Various other hybrid amplification methods such as immuno-PCR can also be used.

The output of DNA amplification system 125 and non-DNA amplification system 135 are both coupled to a biofluidics unit 175, which transports the concentrated samples to biochip 170. The biofluidics unit 175 can be designed using standard solenoid micropumps (Bio-Chem Valve, Inc.), solenoid micro Pinch valves (Bio-Chem Valve, Inc.), syringe pumps (Cavro) or multi-port valves (Cavro). An electronic control system 190 can be used to synchronize all system operations, including sample transport and translation of tape 165.

As noted above, tape 165 provides a renewable supply of microarrays. The microarrays include a plurality of bioreceptor probes preferably representing diverse receptor types, such as DNA, antibody and protein, which can be attached to the surface of a translatable tape 165. Accordingly, tape 165 may be referred to as a multiplex tape 165. Any type of flexible membrane is a generally suitable tape substrate. For example, a commercially available Zeta-Probe membrane provided by Bio-Rad corporation has been used.

Tape production procedures using “printing processes” are known and can be adapted to large scale production at very low cost. The tape 165 surface contains bioreceptors probes which can be arranged in various configurations, such as in parallel tracks, with each track containing a specific type of bioreceptor probes (e.g., antibodies in one track, protein in another track, and DNA in a third track) for a target of interest. The multiplex tape 165 can be mounted on cassettes 168, such that only a portion of the tape (e.g. one microarray) is exposed to sample supplied by biofluidics unit 175 and aligned with the detection biochip 170 at any given time. The cassettes 168 can be sized to fit conveniently into the detection area above the sensor biochip 170 and the optical filter (not shown in FIG. 1) and related optics (not shown in FIG. 1).

The tape 165 is advanced onto the biochip 170 so that the probes of the bioreceptor arrays are aligned with the sensor array of the chip 170. After detection is performed, the tape 165 can be moved forward to align a “new” microarray which comprises a plurality of bioreceptors for a new cycle of detection.

The biochip 170 combines integrated circuit elements including an electrooptics detection system, and bioreceptor probes into a self-contained and integrated microdevice. An excitation source (not shown) such as a laser, can be located on, or off, biochip 170. Example 1 also describes a laser-based illumination system applied to a biosensor system. A data treatment and display (e.g. laptop computer) 195 or an embedded microprocessor can be used to process the data provided by biochip 170.

Biochip 170 preferably includes a CMOS-based sensing array of sensors and related circuitry (e.g. filters, amplifiers, etc.) for converting optical signals, such as Raman, absorption, diffuse reflectance, elastic scattering and fluorescent signals which emanate from a plurality of biochip 170 detection channels, such as photodiodes, phototransistors or avalanche diodes, to electrical signals. Highly integrated biosensors are made possible partly through the capability of fabricating multiple optical sensing elements and microelectronics on a single integrated circuit. With the CMOS technology, highly integrated biosensors are made possible partly through the capability of fabricating multiple optical sensing elements and microelectronics on a single IC.

For example, a compact detection system featuring an integrated circuit (IC)-based 4×4 array detector of independently operating photodiodes has already been demonstrated. The individual photodiodes of the 4×4 array were square with 900-μm edges. The photodiodes were arranged with 1-mm center-to-center spacing. The photodiodes were integrated along with amplifiers, discriminators and logic circuitry on a single platform. The photodiodes and the accompanying electronic circuitry were fabricated using a standard 1.2-μm n-well CMOS process. Other processes and types of sensor arrays may clearly also be used with the invention.

Bioreceptors probes can include DNA, antibody, protein-based probes including enzymes, chemoreceptor, tissue, cells (e.g. microorganism), cell components (e.g. organelle), or biomimetics probes. Bioreceptors generally determine the specificity for biosensor technologies. They are responsible for binding the analyte of interest to the sensor to permit detection and measurement. Bioreceptors can take many forms and the different bioreceptors that have been used are as numerous as the different analytes that have been monitored using biosensors. However, bioreceptors can generally be classified into five different major categories. These categories include: 1) antibody/antigen, 2) enzymes, 3) nucleic acids/DNA, 4) cellular structures/cells and 5) biomimetics.

Significantly, in previous disclosed systems, each bioreceptor category is used exclusively for a given biochip application. Thus, although a microarray may include a plurality of different receptor probes, the probes provided are all within a single category, such as various nucleic acid/DNA sequences.

In contrast, the invention can simultaneous utilizes diverse types of bioreceptors on a single biochip. This novel “hetero-functional” detection capability which is described in application Ser. No. 09/890,047 provides complementary approaches (“quasi-orthogonal”) for detection and identification of diverse target types. Use of multiple receptor probe types for detection of a given target can provide a significantly reduced false alarm rate.

Other detection schemes focus only on a single basic biological principle, such as the use of nucleic acid hybridization to identify a specific sequence of interest, or the highly specific recognition of three-dimensional structure inherent in an antibody-antigen binding reaction. The proposed device, however, can use multiple biological principles to provide information at several tiers of biological identification to increase confidence in positive identification and to decrease the likelihood of false positives.

Detection based upon DNA hybridization is highly specific and theoretically should suffice to unequivocally identify microorganisms, provided the probe sequences are appropriately selected. However, there is always a difference between theory and practice, which can often be significant. For example, there are always possibilities that flaws in the PCR cycling, hybridization conditions, and other sample preparation or reaction conditions can lead to erroneous hybridization and thus errors in identification. However, this uncertainty can be largely eliminated by having an independent set of analytical criteria to provide confirmation of data provided by DNA probes. Thus, use of antibodies to detect highly pathogen-specific antigens of biological warfare (BW) agents, in effect, can provide a second independent assessment of whether a particular BW agent is present or not, thus reducing false alarm rates. This same methodology can be extended to detection of other hazardous materials, such as biological toxins as well as chemical warfare (CW) agents since antibody probes can also generally be designed to detect chemicals.

One type of probe that can be used with the invention is a DNA probe. The operation of gene probes is based on the well known hybridization process. Hybridization involves the joining of a single strand of nucleic acid with a complementary probe sequence. Hybridization of a nucleic acid probe to DNA biotargets, such as gene sequences, bacteria, or viral DNA, offers a very high degree of accuracy for identifying DNA sequences complementary to that of the probe. Nucleic acids strands tend to be paired to their complements in the corresponding double-stranded structure. Therefore, a single-stranded DNA molecule will seek out its complement in a complex mixture of DNA containing large numbers of other nucleic acid molecules. Hence, nucleic acid probe (i.e., gene probe) detection methods are very specific to DNA sequences. Factors affecting the hybridization or reassociation of two complementary DNA strands include temperature, contact time, salt concentration, and the degree of mismatch between the base pairs, and the length and concentration of the target and probe sequences.

Labeled and unlabeled DNA probes can be synthesized as needed, or purchased from a commercial source, such as Oligos Etc., Wilsonville, Oreg. Desired strands of oligonucleotides have been synthesized and labeled with fluorescent labels, such as fluorescein and Cy5 dyes.

Biologically active DNA probes can be directly or indirectly immobilized onto a transducer detection surface to ensure optimal contact and maximum detection. When immobilized onto a substrate, the gene probes are stabilized and, therefore, can be reused repetitively. In the simplest procedure, hybridization is performed on an immobilized target or a probe molecule attached on a solid surface such as a nitrocellulose, a nylon membrane or a glass plate.

Several methods can be used to bind DNA to different supports. The method commonly used for binding DNA to glass involves silanization of the glass surface followed by activation with carbodiimide or glutaraldehyde. One approach used involves silanization for binding to glass surfaces using 3-glycidoxypropyltrimethoxysilane (GOP) or aminopropyltrimethoxysilane (APTS) to covalently link DNA via amino linkers incorporated either at the 3′ or 5′ end of the molecule during DNA synthesis.

Another approach consists of immobilizing the gene probe onto a membrane and subsequently attaching the membrane to the transducer detection surface. This approach avoids the need of binding the bioreceptor onto the transducer and could possibly allow easier large-scale production. Several types of membranes are available for DNA binding, such as nitrocellulose and charge-modified nylon. The gene probe is then bound to the membrane using ultraviolet activation.

The CSP biochip is designed to be compatible to a wide variety of amplification techniques such as polymerase chain reaction (PCR), which is a technique allowing replication of defined DNA sequences, thereby amplifying the detection of these sequences, the strand displacement amplification (SDA) technique [developed by BD Sciences], immuno PCR techniques, and other hybrid techniques.

Another receptor probe type which may be used with the invention is antibody probes. Antibodies are biological molecules that exhibit very specific binding capabilities for specific structures. This is very important due to the complex nature of most biological systems. An antibody is a complex biomolecule, made up of hundreds of individual amino acids arranged in a highly ordered sequence. For an immune response to be produced against a particular molecule, a certain molecular size and complexity are necessary. Proteins with molecular weights greater then 5000 Da are generally immunogenic. The way in which an antigen and its antigen-specific antibody interact may be understood as analogous to a lock and key fit, by which specific geometrical configurations of a unique key enables it to open a lock. In the same way, an antigen-specific antibody “fits” its unique antigen in a highly specific manner. This unique property of antibodies is the key to their usefulness in immunosensors where only the specific analyte of interest, the antigen, fits into the antibody binding site. Antibodies as with other bioreceptors can be immobolized on the tape surface using a variety of standard chemical binding procedures, the procedure selected depending on the nature of the substrates and the particular bioreceptors.

Another probe type which may be used with the invention is enzyme probes. Enzymes are often chosen as bioreceptors based on their specific binding capabilities as well as their catalytic activity. In biocatalytic recognition mechanisms, the detection is amplified by a reaction catalyzed by macromolecules called biocatalysts. With the exception of a small group of catalytic ribonucleic acid molecules, all enzymes are proteins. Some enzymes require no chemical groups other than their amino acid residues for activity. Others require an additional chemical component called a cofactor, which may be either one or more inorganic ions, such as Fe2+, Mg2+, Mn2+, or Zn2+, or a more complex organic or metalloorganic molecule called a coenzyme. The catalytic activity provided by enzymes allows for much lower limits of detection than would be obtained with common binding techniques.

Other probe types which may be used with the invention include cells or cell components, and biomimetics. Biomimetics can include molecular imprint antibodies, DNA-based aptamers, PNA, cyclodextrins and dendrimers.

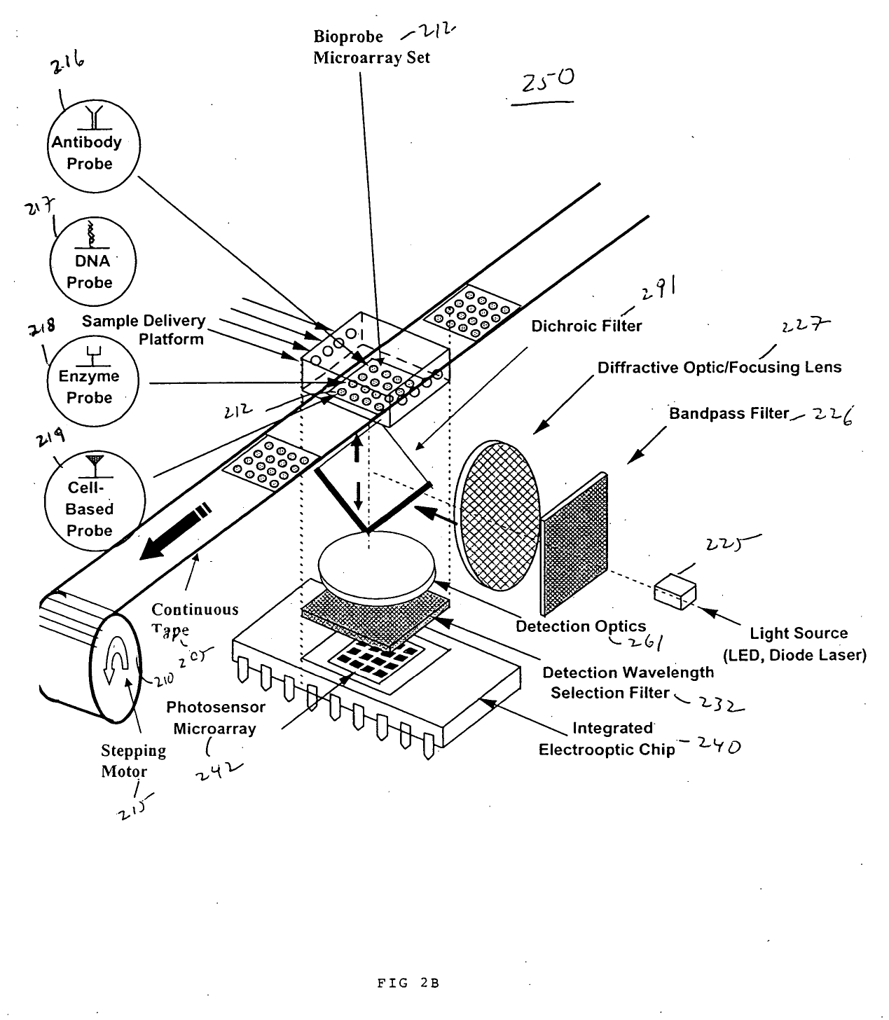

FIG. 2A shows an exemplary biochip system 200 embodiment. A tape 205 is drawn from a roll 210 through a sample delivery platform 222 using a stepping motor 215. The tape 205 includes a series of microarrays of bioreceptor probes 212 which comprise antibody probes 216, DNA probes 217, enzyme probes 218 and cell-based probes 219 which are disposed on the surface of tape 205 and are thus outwardly exposed. The tape 205 follows a path defined by a series of go-and-stop cycles determined by the detection-probe exposure (e.g., DNA hybridization or antibody/antigen binding) cycles within the sample delivery platform 222.

A source of analyte such as a biofluidics-based unit (not shown) can deliver liquid samples of processed and/or amplified samples (e.g., amplified DNA following PCR, or amplified products following ELISA reaction) into the sample delivery platform 222 where the DNA hybridization and/or antibody-antigen binding can occur at the probes provided by microarray 212. Each bioreceptor probe microarray 212 is shown including sixteen (16) receptor probes 216-219. The sample delivery platform 222 and the stepping motor 215 can be interfaced with a microprocessor (not shown) which is programmed to control the speed of the tape 205 and the sample delivery, and sample-probe interaction time intervals.

A heating/cooling device (e.g. thermoelectronic Peltier chip) can provide thermal control of the reactions inside the sample delivery platform housing 222. The tape is aligned such that each set of microarray probes 212 is excited by light from light source 225, such as an LED or laser, after passing through optional bandpass filter 226 and being diffracted by diffracting optic/focussing lens 227. Diffracting optic/focussing lens 227 can provide a plurality of excitation light beams, such as sixteen (16) to provide one light beam per probe, the respective light beams having an area to match the area of the respective receptor probes on microarray 212. Reflective optic 229 directs the light beams produced by diffracting optic/focussing lens 227 towards microarray probes 216-219.

Assuming fluorescent spectroscopy is used, the resulting fluorescence signals produced if binding events take place at respective probes 216-219 are directed via the GRIN lens array 231 toward integrated electrooptic chip 240. A detection wavelength selection filter 232 preferably is included to isolate the fluorescent signal of interest and to eliminate background signals as well as the laser (or LED) scattered light. Following wavelength selective filtering, the fluorescent signals reaches integrated biochip 240.

Biochip 240 includes integrated electrooptics, such as a photosensor microarray 242 based on an array of optoelectronic transducers, such as photodiodes, phototransistors or avalanche diodes. As shown in FIG. 2, the photosensor microarray includes sixteen (16) sensors, one for each receptor probe on microarray 212. This arrangement permits each detection channel to have customizable characteristics to match the associated bioreceptor, such as high gain for normally low signal levels. Although generally preferable to have one sensor for each receptor probe, the invention can clearly be practiced with an unequal number, such as possible through use of a multiplex switch.

FIG. 2B shows a system 250 which is substantially similar to system 200, with like components having like reference numbers, except a dichroic filter 291 is utilized. Dichroic filter 291 reflects light emitted by light source 225 to microarray 212. Red shifted fluorescent light emanated from probes 216-219 is transmitted by dichroic filter 291 through optics 261 to photosensor array 242 for detection. Dichroic filter 291 is preferred to a band pass filter since dichroic filters are generally far more accurate and efficient in their ability to block unwanted wavelengths as compared to gel or glass absorption filters.

Placement of light source 225 between sampling platform 212 and biochip 240 in system 250 permits system 250 to be substantially more compact as compared to system 200. Moreover, this arrangement can reduce light attenuation as compared to system 200.

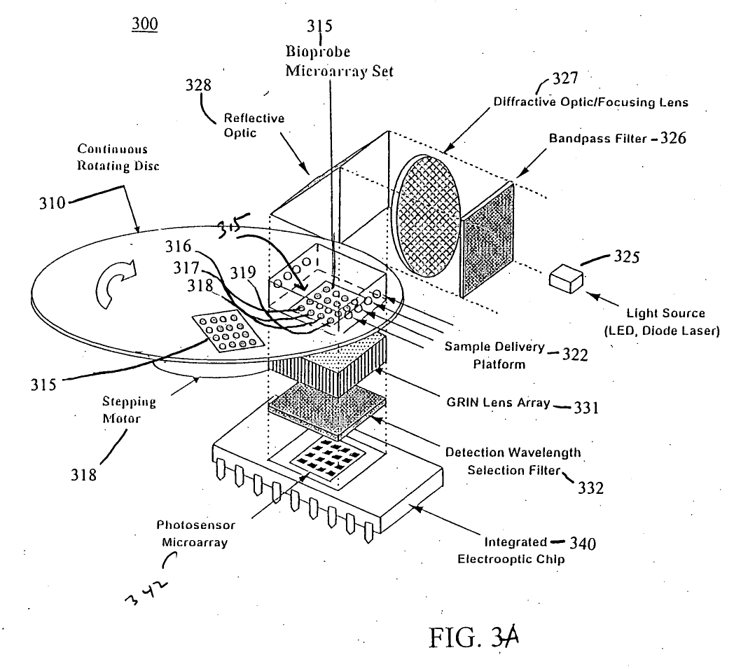

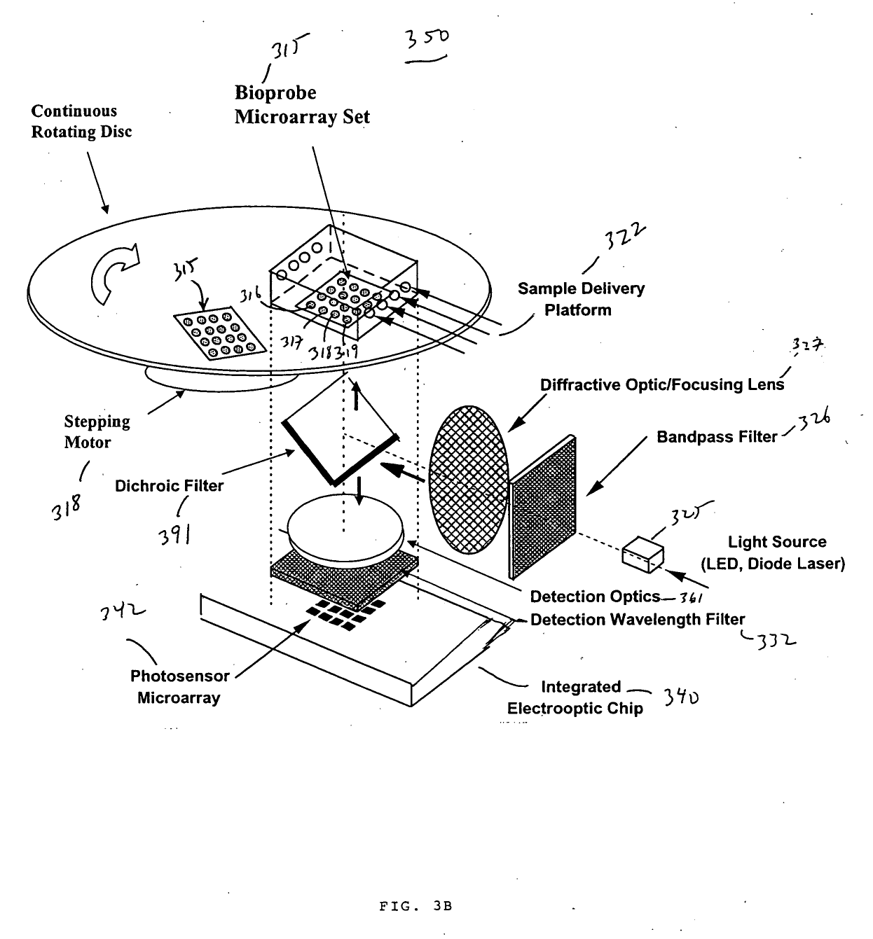

FIG. 3A shows a biochip system 300 which includes a rotating disc 310 which provides a plurality of probe microarrays 315, each microarray 315 having a plurality of receptor probes 316-319. The disc 310 is mounted on a rotating platform (not shown) driven by a stepping motor 318, such that only a portion, such as one microarray, of disc 310 is rotated through the sample delivery platform 322 and exposed and aligned to the detection chip 340 at any given time. Once a detection cycle is performed, the disc 310 can be rotated so that successive portions of the disc 310 with a new set of microarray probes 316-319 are aligned above the integrated electrooptic chip 340.

The disk 310 is aligned such that each set of microarray probes 315 is excited by light from light source 325, such as a LED or laser, after passing through optional bandpass filter 326 and being diffracted by diffracting optic/focussing lens 327. Diffracting optic/focusing lens 327 can provide a plurality of excitation light beams, such as sixteen (16) to provide one light beam per probe 316-319, the respective light beams having an area to match the area of the respective receptor probes on microarray 315. Reflective optic 328 directs the light beams produced by diffracting optic/focussing lens 327 towards probes on microarray 315.

Assuming fluorescence spectroscopy is used, the resulting fluorescence signals produced if binding events take place at respective probes on miroarray 315 is directed via the GRIN lens array 331 toward integrated electrooptic chip 340. A detection wavelength selection filter 332 preferably is included to isolate the fluorescence signal of interest and to eliminate background signals as well as the laser (or LED) scattered light. Following wavelength selective filtering, the fluorescence signals reaches electrooptic chip 340. Electrooptic chip 340 includes a plurality of electroptic sensors, one for each probe on microarray 315.

The sample delivery platform 322 and the stepping motor 318 can be interfaced with a microprocessor (not shown). The microprocessor can be programmed to control the speed of rotation of disc 310, sample delivery, and sample-probe interaction time intervals.

FIG. 3B shows a system 350 which is substantially similar to system 300, with like components having like reference numbers, except a dichroic filter 391 is utilized. Dichroic filter reflects light from light source 325 to microarray set 315. Red shifted fluorescent light emanated from probes 316-319 is transmitted by dichroic filter 391 through optics 361 to photosensor array 342 for detection.

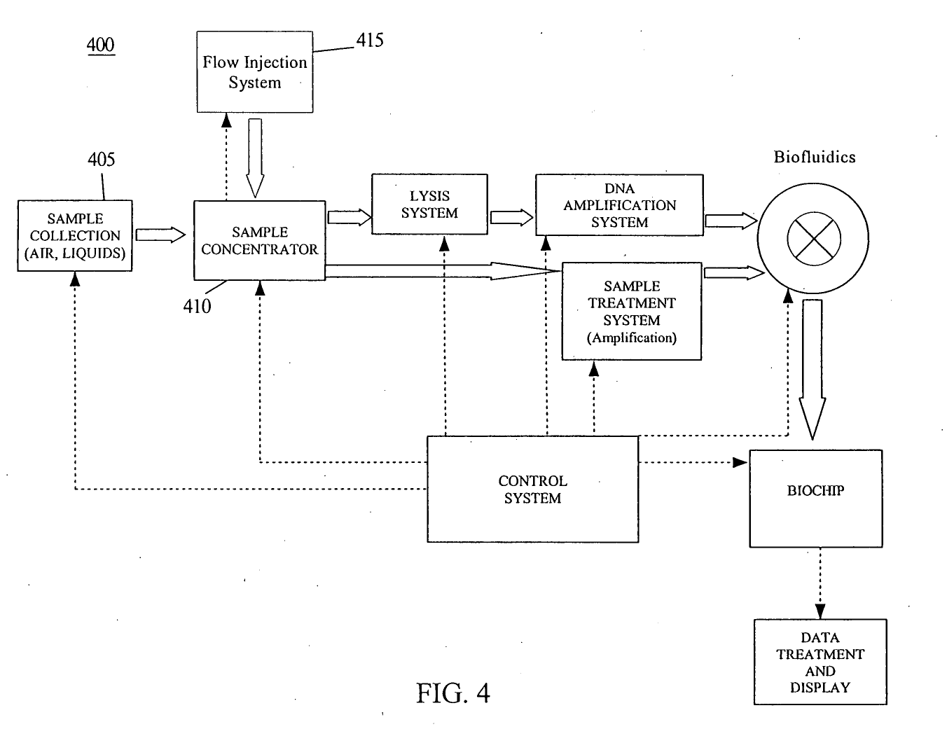

FIG. 4 shows a bloc diagram of an integrated biochip based system 400 including a sample concentrator 410 based on a flow injection assay system (FIA) 415. Other than the presence of flow injection assay system 415, system 400 is otherwise identical to system 100 shown in FIG. 1.

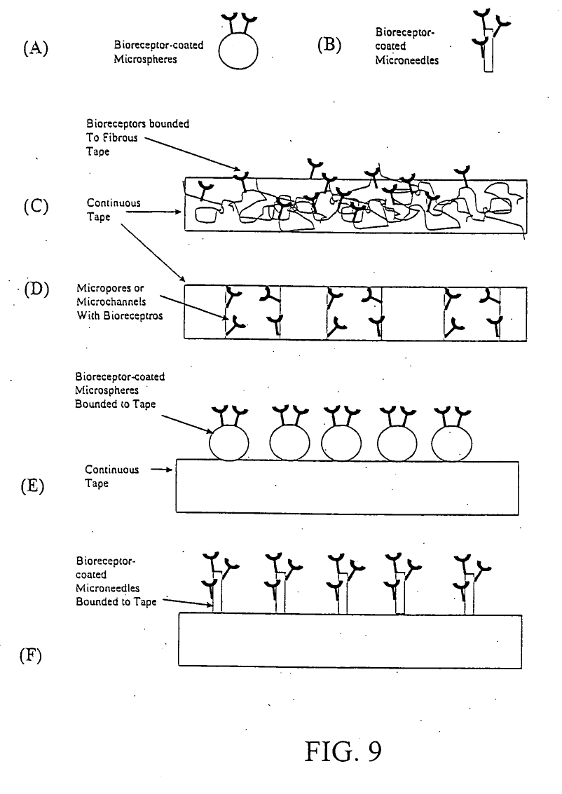

Flow injection analysis (FIA) system 415 is used to introduce microparticles (e.g., microbeads, micro-needles, see FIG. 9A-F) or nanoparticles (e.g. nanobeads, nanoneedles) coated with a bioreceptors (e.g., antibodies) targeted to one or more species of interest into sample concentrator 410 along with sample collected at sample collector 405. The sample is concentrated because bioreceptor microparticles will bind only to the species of interest and thus can be used to remove the compound of interest from a sample which comprises a complex mixture of species. In previous work flow injection assay (FIA) devices with microbeads coated with antibodies in a fiberoptic biosensor device have been used to concentrate samples [Refs: J. P. Alarie, J. R. Bowyer, M. J. Sepaniak and T. Vo-Dinh, “Fluorescence Monitoring of Benzo(a)pyrene Metabolite Using a Regenerable Immunochemical-Based Fiberoptic Sensor,” Anal. Chim. Acta., 236, 237 (1990); T. Vo-Dinh, M. J. Sepaniak, G. D. Griffin, and J. P. Alarie, “Immunosensors: Principles and Applications,” Immunomethods, 3, 85 (1993); M. J. Sepaniak and T. Vo-Dinh, “Fiber Optic-Based Regenerable Biosensor,” U.S. Pat. No. 5,176,881 (1993)]. Microbeads systems have also been used in systems for column-based separations, methods of forming packed columns, and methods of purifying sample components [O. B. Egorov, M. 0 Hara, J. W. Grate, D. P. Chandler, F. J. Brockman, C. Bruckner-Lea, U.S. Pat. No. 6,136,197, 2000)]. In a preferred embodiment, the bioreceptor microparticles provide multifunctional sensing, such as by providing DNA, protein and antibody based bioreceptor.

The FIA based system 400 can comprise of a plurality of capillary columns (not shown), which function to deliver microparticles coated with bioreceptors (e.g., immunobeads, or micro-needles coated with bioreceptors) or liquid reagents, or rinse solution as needed. Each capillary can be secured in an adapter that includes an on-off valve to facilitate connection to a micropump (not shown) for reagent delivery.

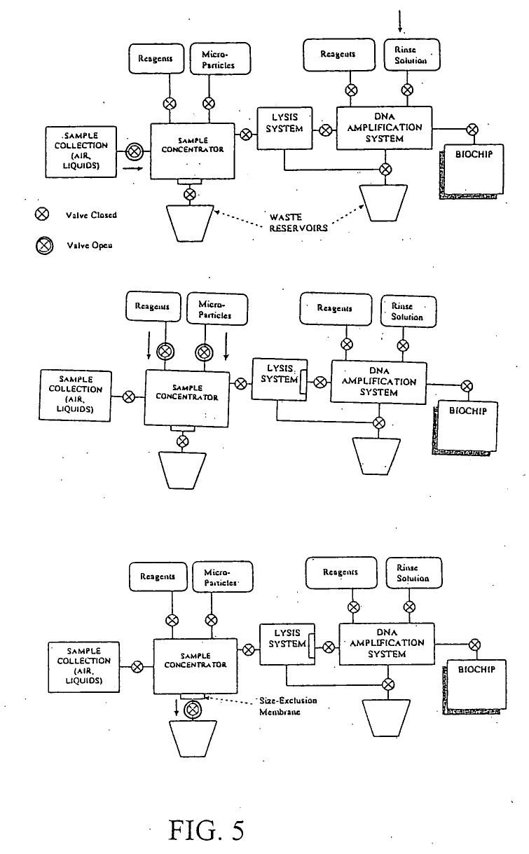

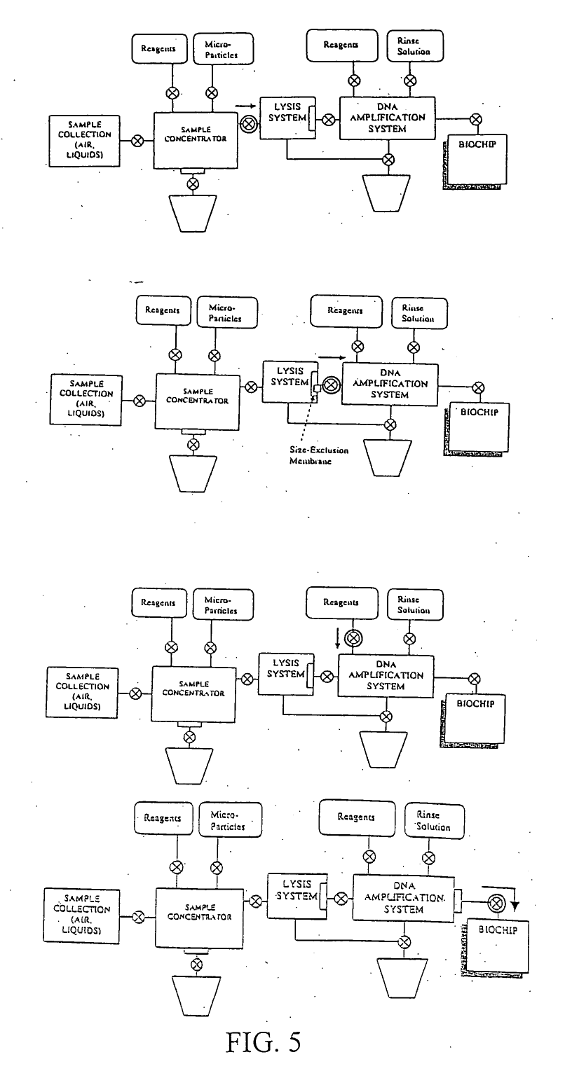

The various steps in an exemplary FIA based CSP biochip are illustrated in FIG. 5. In step 510 the FIA system aspires the liquid sample extract from the air sampler (e.g. Mesosystems Technology, Inc.) outlet into the sample concentration chamber. The FIA system then introduces the bioreceptor-coated microparticles (e.g., immunobeads) into the concentrator to permit binding of the target compounds onto the bioreceptors which are bound to the microparticles in step 520. In step 530 an aspiration system comprised of a size-selective membrane or a stainless-steel frit (Newmet Krebsoge) directs substances not trapped onto the microparticles into a waste reservoir (step 530). The microparticles, which contain the species of interest bound to the bioreceptors are larger than the holes of the membrane or the frit, and therefore remain in the sample concentrator.

In step 540 a second aspiration system moves the microparticles from the sample concentrator into the lysis system where the target DNA species from the sample are lysed from the bioreceptor coated microparticles. From this lysing step, in step 550 the DNA and associated microparticles can be sent to a waste reservoir while the DNA target released by the lysing system can be sent to the DNA amplification system, while other desired targets can be sent to another amplification system. In step 560 reagents for amplification are then delivered to the amplification system. Finally the amplified DNA sample is sent to the biochip for detection in step 570.

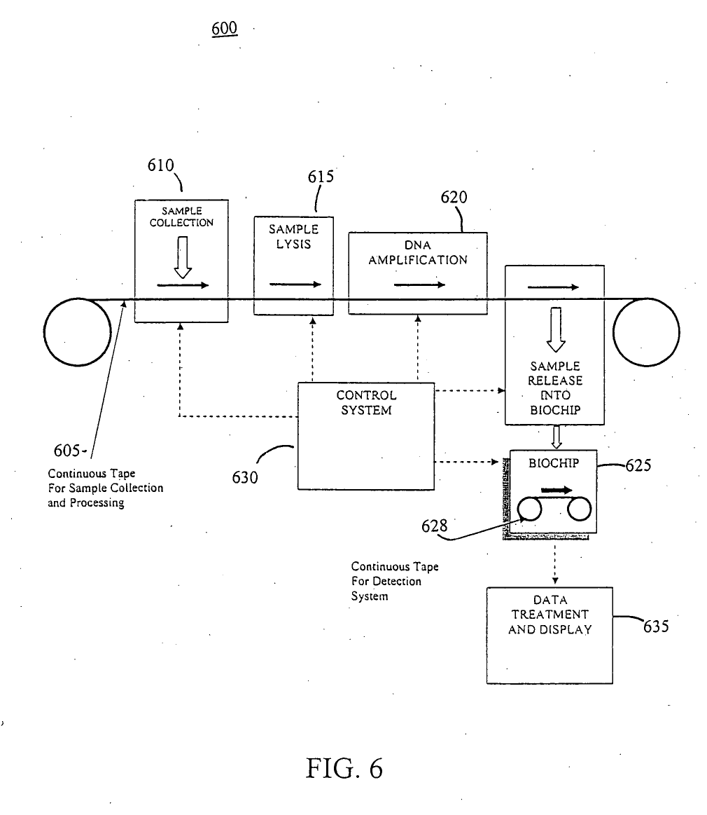

FIG. 6 shows a block diagram of a biochip based system 600 which provides a continuous regenerable tape system including two (2) continuous tapes. A first tape 605 provides sampling collection and processing. A second continuous tape 628 provides detection. Tape 605 provides a surface for sample collection that is newly regenerable, while tape 628 provides a multichannel tape including various bioreceptors for simultaneous detection of different species.

The moving tape 605 enters a sample collection chamber 610. Tape 605 preferably provides bioreceptors that trap particulates from the air or from liquid samples. Bioreceptors can include antibodies, proteins, enzymes, chemicals that can selectively trap species of interest. Following this collection phase, the tape enters a sample lysis chamber 615.

Since the multi-functional biochip can detect both DNA as well as proteins and other target types, the sample collected on the tape 605 is processed in both the DNA channel and the non DNA channel. In the DNA-based channel, the sample on the tape, which contains bioagents (entire organisms, cells, spores, etc) is lysed in the lysis system 615. The lysis system 615 can use heat, chemical, acoustic (ultrasound) or electronic (plasma production by electrodes) to lyse the cells and release the target DNA from the tape 605. The cellular DNA target is then amplified in DNA amplification system 620 which can use polymer chain reaction (PCR) or other amplification techniques (e.g., strand displacement amplification (SDA). The amplified DNA is then sent to the biochip 625 for detection.

An electronic control system 630 is preferably used to synchronized all system 600 operations. A data treatment and display 635 is can be included to process the data.

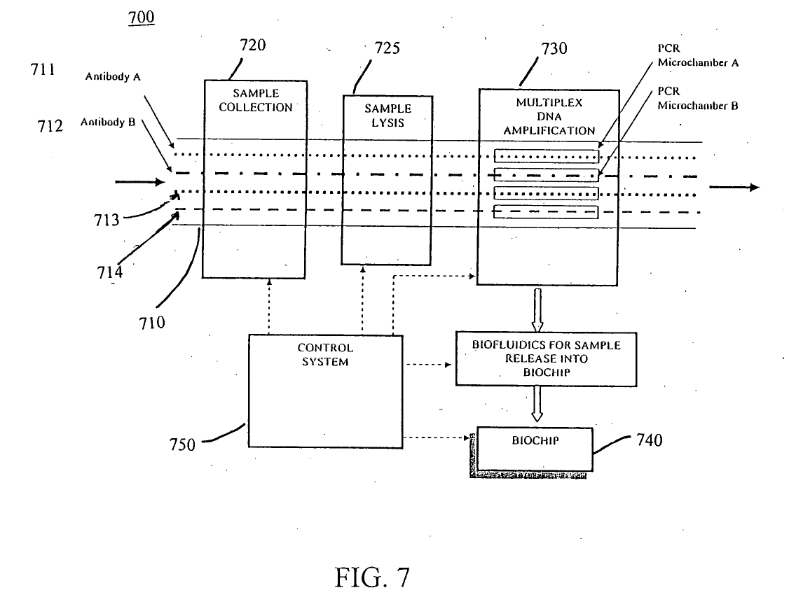

FIG. 7 shows a multiplex tape system 700 for continuous sample collection and processing. The tape 710 shown is designed to contained multiple tracks of diverse bioreceptor types, such as antibodies targeted to bind to specific targets. For example, antibodies A 711 for bacteria A and antibodies B 712 for virus B, proteins C 713 for cells C, biomimetic receptor D 714 for agents D. The tape 710 enters sample collection chamber 720 where each track of bioreceptors 711-714 collects and concentrates targeted agents and extracts them from the sample mixture if present. The sample mixture can be a liquid sample or a liquid extract of an air sample. In this process multiple targets are concentrated simultaneously in a multiplex fashion.

The portion of tape 710 containing target species trapped by the biorepceptors 711-714 enters the sample lysis module 725 where agents contained in each track are lysed simultaneously in each track. Each track preferably has a separated microchamber (not shown). The tape 710 then enters a multiplex DNA amplification chamber 730, which contain separate microchambers where each PCR operation is performed using the temperature cycling or other lysing conditions optimized to each species of interest. For non-DNA channels, there is no need for a lysis system (see FIG. 1). Accordingly, the amplification chamber is designed for non-amplification, such as ELISA (enzyme-linked immunosorbent assay). Following amplification, a biofluidics unit 735 preferably carries the sample to biochip 740 for detection. A control system 750 can control the operations of system 700.

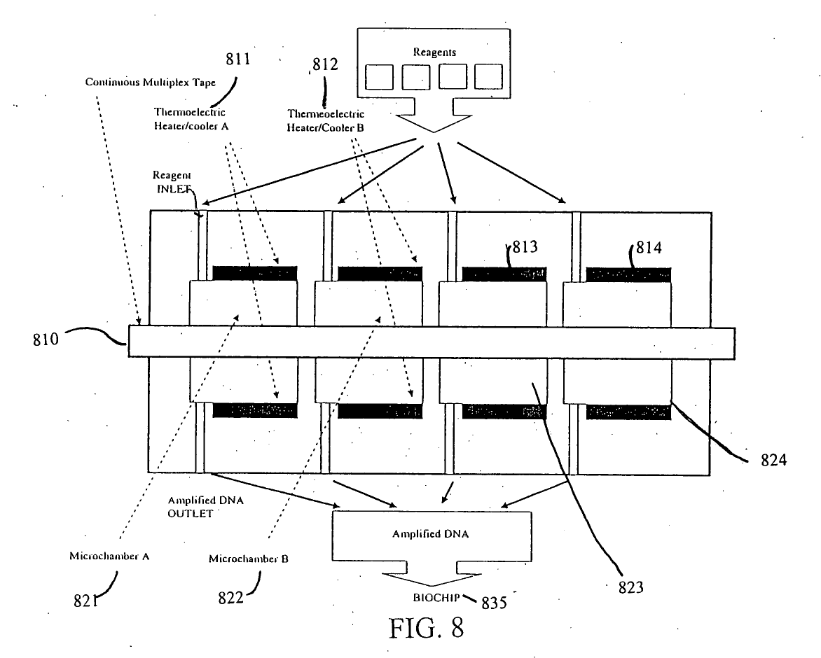

A vertical cross sectional view of a multiplex PCR multi-microchamber system 800 is shown in FIG. 8. The tape 810 has 4 tracks each containing a different antibody for binding to a specific target. The tape 810 is translated in a direction that is normal to the drawing surface. A biofluidic system (not shown) delivers reagents to microchambers 821-824 through respective reagent inlets. Each chamber has a set of different thermoelectric blocs 811-814 (e.g. Advanced Thermoelectrics), which are set for a specific thermal cycling temperature conditions optimized for the species of interest.

In this embodiment, 4 different species can be amplified at the same time. The DNA targets of interest are amplified and can be labeled with fluorescent labels in the same operation using standard procedure in PCR. The method involves using a fluorescent-labeled DNA sequence as a primer in PCR amplification of the target DNA followed by hybridization to the capture probe sequences bound to the continuous tape. The capture probes are complementary to an internal sequences of the target DNA (and of the amplified products). Finally all the labeled and amplified DNA target segments are released to the biochip 835 for detection.

The microparticle-based sampler can include microspheres, microbeads or microneedles coated with bioreceptors. FIG. 9A shows bioreceptor coated microspheres, while FIG. 9B shows bioreceptors coated microneedles.

The continuous tape is preferably a flexible material that can hold several different bioreceptor structures. A membrane with fibrous structure having bioreceptors bound to the fibrous tape is shown in FIG. 9C. The fibrous woven fibers provide the 3-dimensional increase in surface area. Therefore, an increased number of bioreceptors can be bound as compared to a planar tape.

A membrane tape having microchannels is shown in FIG. 9D. The tape contains micropore holes and microchannels that provide preferred sites for binding bioreceptors. FIG. 9E shows a membrane comprising microparticles with bioreceptor coated biospheres attached to its surface. The microparticles provide increased surface areas for binding bioreceptors. Note that magnetic microbeads can be used and will allow transport by using magnetic fields. FIG. 9F shows a membrane that contains microneedles coated with bioreceptors. The microneedles provide increased surface areas for binding bioreceptors. Magnetic microparticles and microneedles can be used which allow transport aided by magnetic fields.

The broad-based biosensing capability provided by the invention can be used within stationary indoor and outdoor field sites as well as on mobile platforms for early warning and human health protection. The system can also be designed as a portable personal monitor as well as an area monitor for use in civilian facilities (e.g., office buildings, subways) remote from analytical laboratories, which often impose severe constraints on available manpower, equipment, and biochemical supplies for effective detection.

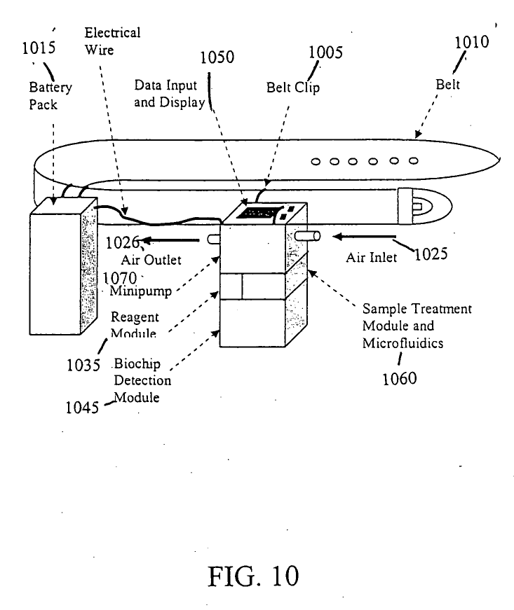

FIG. 10 shows a schematic diagram of an exemplary personal integrated CSP biochip system 1000 which can be conveniently carried by an individual. Personal biochip 1000 can be miniaturized (handheld size) and can serve as a personal monitor for continuous, automatic and real time (or near real time) detection of species of interest in the environment. The biochip can be mounted to a belt 1010, such as by belt clip 1005. A battery pack 1015 can provide the energy needed to power the various components of biochip system 1000. The battery pack 1015 is preferably a high energy density secondary battery, such as a lithium ion or lithium metal based battery.

The personal device 1000 consists of an air sampler having an air inlet 1025 and air outlet 1026, a sample treatment module with associated microfluidics 1060 which along with minipump 1070 provides sample concentration processing. The reagent module 1035 delivers the reagents and bioreceptors required for the assays.

The processed sample is then fed into the biochip module 1045 for detection. For example, biochip module 1045 can be based on a continuous tape which provides a plurality of receptor microarrays as shown in FIG. 2A or 2B, or a spinning disk which provide a plurality of receptor microarrays as shown in FIG. 3A or 3B. Upon detection of target analytes, personal biochip 1000 can provide a visual (e.g. blinking light) display 1050 and/or an audible alarm.

CSP biochip systems can be used for many other applications which can benefit from autonomous and rapid sensing of a wide variety of chemical and biological (CB) substances. For example, the invention can be used to support of monitoring activities related to homeland defense, non-proliferation and terrorist prevention activities, verification and monitoring of non-compliance production facilities for CB, automated analysis of pharmaceuticals, and high-throughput drug screening and related activities. The invention can also be used for continuous analysis of food and agricultural products and continuous environmental monitoring including air quality monitoring.

### EXAMPLES

The present invention is further illustrated by the following specific examples. The examples are provided for illustration only and are not to be construed as limiting the scope or content of the invention in any way.

### Example 1

### Exemplary Illumination System

A HeNe laser (Model 106-1, Spectra-Physics, Inc., Eugene, Oreg.) or a diode laser (Process Instrument) was selected for excitation of the Cy5 label (632.8 nm). The laser beam was filtered with a 632.8-nm bandpass filter (Cat. No. P3-633-A-X516, Corion, Franklin, Mass.) and directed through a diffractive pattern generator, which produced a 4×4 array of equally intense laser excitation spots which were directed onto a microdot-encoded membrane. The intensity of each laser spot was estimated to be approximately 0.2 mW. Proper distance between the pattern generator and the microdot array platform was used to generate approximately 1-mm spacing between the laser excitation spots.

The microdot array printed on the membrane was aligned with the focused laser excitation spot array. Incorporation of visible microdots in the four corners of the printed microdot array pattern facilitated this alignment. A 1:1 image of the laser spot array was projected from the microdot array-encoded membrane onto the corresponding 4×4 array of photosensors of the IC detector via a gradient index microlens array (Cat. No. 024-5680, OptoSigma®, Santa Ana, Calif.). A combination of a 633-nm holographic notch filter (Cat. No. HNPF-633-1.0, Kaiser Optical Systems, Inc., Ann Arbor, Mich.) and a thin-film dielectric filter with a high-pass at 645 nm (Visionex, Atlanta, Ga.) was used to isolate the Cy5 emission signal from the excitation laser line. Voltage output from the IC biochip was recorded from a digital multimeter (Model 506, Protek).

### Example 2

### Synthesis, Labeling and Immobilization of DNA and Antibody Probes

Laboratory-prepared oligonucleotides were synthesized using an Expedite 8909 DNA synthesizer (Millipore). Oligonucleotides with amino linkers were synthesized using either C3 aminolink CPG for 3′ labeling or 5′ amino modifier C6 (Glenn Research, Sterling, Va.) for 5′ labeling. All oligonucleotides were synthesized using Expedite reagents (Millipore) and were de-protected and cleaved from the glass supports using ammonium hydroxide. The de-protected oligonucleotides were concentrated by evaporating the ammonium hydroxide in a Speedvac evaporator (Savant) and resuspended in 100 μL distilled H₂O. Further purification was performed by isopropanol precipitation of the DNA as follows: 10 μL of 3-M sodium acetate pH 7.0 and 110 μL isopropanol was added to 100 μL solution of DNA. The solution was then frozen at −70° C. The precipitate was collected by centrifugation at room temperature for 15 min and was washed 3 times with 50% isopropanol. Residual isopropanol was removed by vacuum drying in the Speedvac and the DNA resuspended in sterile distilled water at a final concentration of 10 μg/μL. These stock solutions were diluted in the appropriate buffer at a 1:10 dilution to give a DNA concentration of 1 μg/μL.

To label DNA with the near-infrared (NIR) Cy5 dye (Amersham Life Sciences, Arlington Heights, Ill.) modified oligonucleotides containing alkyl amino groups were derivatized as follows: 30 pmoles of the DNA was dissolved in 250 μL 0.5-M sodium chloride and passed through a Sephadex G10 (1 cm diameter, 10 cm long) (Pharmacia, San Diego, Calif.) column equilibrated with 5 mM borate buffer (pH=8.0). The void volume containing the oligonucleotides was collected and concentrated by evaporation. The resulting solution was dissolved in 100 μL 0.1-M carbonate buffer (pH=9.0). Cy5 (1 mg in carbonate buffer) was added to the oligonucleotides and the conjugation reaction was performed at room temperature for 60 min with occasional mixing. The conjugated oligonucleotide was separated from the free dye using a Sephadex G10 column as described above. The fractions containing the labeled DNA were collected and concentrated using a Speedvac evaporator.

Several methods have been investigated to bind DNA to different supports that can be used as materials for the biochip sampling platform. One method for binding DNA to glass involved silanization of the glass surface followed by activation with carbodiimide or glutaraldehyde. Silanization methods were initially used for binding to glass surfaces using 3-glycidoxypropyltrimethoxysilane (GOPS) or aminopropyltrimethoxysilane (APTS) and attempted to covalently link DNA via amino linkers incorporated either at the 3′ or 5′ end of the molecule during DNA synthesis. Another approach used involved binding the DNA probe onto a membrane and subsequently attaching the membrane directly to the transducer detection surface. This approach avoids the need of binding the bioreceptor onto the transducer and could possibly allow easier large-scale production. Several types of membranes were available for DNA binding including nitrocellulose and charge-modified nylon. The DNA probe was then bound to the membrane using ultraviolet activation.

Arrays of DNA probes were produced on the sampling platform by spotting (placing) the DNA on Immunodine-ABC nitrocellulose membrane using a pV 830 pneumatic Picopump (World Precision Instruments, Sarasota, Fla.). Fluorescence measurements of the hybridized DNA were performed using the biochip using an appropriate laser excitation (diode laser or a He-Ne, Melles Griot).

Several methods for preparing and labeling of antibody probes were investigated. In one investigation, arrays of antibody to wild type human p53 or rabbit anti goat IgG (Sigma chemical company, St. Louis, Mo.), were produced on the HFB sampling platform by spotting on the immunodyne ABC nitrocellulose membrane (Pall Corporation, East Hills, N.Y.) using a pneumatic Picopump (World Precision Instruments, Sarasota, Fla., model pV 830) which was programmed to deliver arrays of microspots of desired formats. The human blocking peptide to p53 was purchased from Santa Cruz Biotechnologies (Santa Cruz, Calif.). Labeling of the peptide with the Cy5 dye was performed using the following protocol. The peptide was dissolved in 0.1-M sodium carbonate, bicarbonate buffer (pH 9.3) to the final concentration of 1 mg/ml. One ml of this antigen solution was added to the Cy5 labeling dye vial (Fluorolink Cy-5 Reactive Dye Pack, Biological Detection Systems, Inc., Pittsburgh, Pa.) and incubated for 30 min at room temperature. Following the dye conjugation step, the labeled peptide was separated from the free dye using a Sephadex G-50 column and eluting the mixture with phosphate buffered saline (pH 7.4). Fractions corresponding to the faster moving (the labeled protein) were collected and pooled.

Arrays of antibody to wild type human p53 or rabbit anti goat IgG (Sigma chemical company, St. Louis, Mo.), were produced on the HFB sampling platform by spotting on the immunodyne ABC nitrocellulose membrane (Pall Corporation, East Hills, N.Y.) using a pneumatic Picopump (World Precision Instruments, Sarasota, Fla., model pV 830) which was programmed to deliver arrays of microspots of desired formats.

### Example 3

### Microarray Spotting Using Thermal Printing Procedures

A commercial color ink-jet printer (Hewlett-Packard Deskjet 694C) was used with a modified color ink cartridge, altered to dispense biological materials. The color cartridge (HP 51694A, Hewlett Packard) consists of three separate ink reservoirs connected by channels to three independent arrays of nozzles in the printing head. The modifications made to the ink cartridge for dispensing of biological materials are detailed below. First, the snap-on plastic top of the cartridge was removed and the internal sponges, made of polyurethane foam and soaked with cyan, magenta, and yellow inks, were removed and discarded. Next, the circular metal screens attached to the bottom of each ink reservoir, used to filter solid particles and break air bubbles, were removed, exposing the ink channels. After removing the metal screens, the cartridge was washed with distilled water and ethanol several times, until all ink was removed, and air-dried. Finally, the biological samples were directly inserted into the ink channels with a micropipette. The printer was capable of working with a low volume (60 mL per channel) of biological samples, and the cartridges could be washed with water and ethanol numerous times with no loss of printing quality.

In order to improve microjet spotting, 10 L of ethanol was added to 100 L of each DNA extraction solution. The individual reservoirs of the three-color cartridge from a conventional thermal ink-jet printer (HP 694C) were filled with 60 L of the biological material solution, and printed onto a Zetaprobe membrane forming a 16-element matrix pattern (FIG. 1B). Zetaprobe membranes were chosen since they have a positively charged surface that electrostatically adsorbs DNA and other anionic macromolecules. Using the modified cartridges, several arrays (in a 4′4 matrix format) were printed with the highest resolution settings of the printer.

After spotting, DNA was immobilized on the membrane by exposure to UV for one minute. The membrane was then blocked in 5 mL of the prehybridization solution for 1 h at 37° C. (5×SSC, 1% non-fat dry milk, and 0.02% sodium dodecyl sulfate (SDS)). The Cy5-labeled probes to FHIT DNA (Oligos etc. Wilsonville, Oreg.) were added to the pre-hybridization solution at 100 ng/mL each and incubated at 37° C. for 16 h. Before detection, the membrane was washed in 5 mL of wash solution containing: 5SSC, and 0.1% SDS for 15 min at room temperature, followed by two 1-minute rinses with water.

While the preferred embodiments of the invention have been illustrated and described, it will be clear that the invention is not so limited. Numerous modifications, changes, variations, substitutions and equivalents will occur to those skilled in the art without departing from the spirit and scope of the present invention as described in the claims.

1. An integrated circuit based detection system, comprising:
a plurality of probe microarrays, each of said microarrays having a plurality of receptor probe elements for combining with at least one target molecule;
a source of electromagnetic radiation, said probe elements generating an identifiable signal when combined with said target molecule in response to said electromagnetic radiation;
structure for translating said plurality of microarrays, wherein said microarrays are replaceable by others of said plurality of microarrays, and
an integrated circuit microchip including a plurality of detection channels to which said probe elements are brought into optical alignment for sensing the presence of said target molecule based on said signal.

2. The system of claim 1, wherein at least one of said microarrays comprise at least one protein probe and at least one nucleic acid probe.

3. The system of claim 1, wherein said microarrays comprise at least two probes selected from the group consisting of DNA, RNA, antibodies, proteins, enzymes, cells or cell components, and biomimetics.

4. The system of claim 3, wherein said biomimetics are at least one selected from the group consisting of molecular imprint antibodies, DNA-based aptamers, PNA, cyclodextrins and dendrimers.

5. The system of claim 1, further comprising an air sampler for collecting airborne samples.

6. The system of claim 1, wherein said system comprises a sample concentrator.

7. The system of claim 6, wherein said sample concentrator comprises a flow injection analysis system.

8. The system of claim 7, wherein said flow injection analysis system comprises a plurality of microparticles coated with bioreceptors, said coated microparticles mixed with a sample at said sample concentrator.

9. The system of claim 8, wherein said concentrator includes a size exclusion device for eliminating substances not trapped onto said coated microparticles.

10. The system of claim 1, wherein said system further comprises a biofluidics system having a plurality of microfluidic channels, said biofluidics system for directing samples through said microfluidic channels to said microarrays.

11. The system of claim 1, wherein said plurality of microarrays are provided on a translatable tape.

12. The system of claim 11, wherein said system further comprises structure for translating said tape.

13. The system of claim 1, wherein said microarrays are provided on a rotable disk.

14. The system of claim 1, wherein said integrated circuit microchip provides a separate detector channels for each of said receptor probes on said microarrays.

15. The system of claim 14, wherein detectors for said detector channels are selected from the group consisting of photodiodes and phototransistors.

16. The system of claim 1, wherein a continuous tape having said plurality of miccroarrays provides sample collection and processing.

17. The system of claim 1, further comprising a target amplification system.

18. The system of claim 17, wherein said target amplification system comprises at least one selected from the group consisting of a PCR, SDA, ELISA and immuno-PCR.

19. The system of claim 1, further comprising a lysis system.

20. The system of claim 1, further comprising an audio or visual display to indicate the presence of said target molecule.

21. The system of claim 20, further comprising structure for attaching said system to an individual.

22. A method of detecting target analytes, comprising the steps of:
providing a plurality of probe microarrays, each of said microarrays having a plurality of probe elements for combining with at least one target molecule, wherein said probe elements generate an identifiable signal when combined with said target molecule in response to electromagnetic radiation;
exposing probe elements on a first of said plurality of microarrays to a sample suspected of containing said target;
irradiating said first microarray with electromagnetic radiation;
determining whether said target is present;
automatically replacing said first microarray with another of said plurality of microarrays, and
repeating said exposing, irradiating and said determining step with said another of said plurality of microarrays.

23. The method of claim 22, wherein at least one of said microarrays comprise at least one protein probe and at least one nucleic acid probe.

24. The method of claim 22, further comprising the step of concentrating said sample.

25. The method of claim 24, wherein said sample concentrating comprises mixing a plurality of microparticles coated with a bioreceptors with said sample.

26. The method of claim 25, further comprising the step of removing substances not trapped onto said coated microparticles.

27. The method of claim 22, wherein said plurality of microarrays are provided on a translatable tape or a rotatable disk.

28. The method of claim 27, further comprising the step of translating said tape or rotating said rotatable disk.

29. The method of claim 22, wherein a continuous tape having said plurality of miccroarrays provides collection and processing for said sample.

30. The method of claim 22, further comprising the step of amplifying a concentration of said target.

31. The method of claim 30, wherein said target is a non-DNA target and said amplification comprises ELISA.

32. The method of claim 22, further comprising the step of lysing said sample.

33. The method of claim 22, further comprising the step of generating an audio or visual alarm to indicate the presence of said target.
